(19) United States
(12) Patent Application Publication
(10) Pub. No.: US 2003/0021825 A1
(43) Pub. Date: Jan. 30, 2003

(54) Cleaning of medical devices with supercritical fluids

(76) Inventors: Chandrashekhar P. Pathak, Austin, TX (US); Joseph A. Chinn, Austin, TX (US); Randall J. Thoma, Austin, TX (US)

Correspondence Address: Timothy L. Scott, Senior Intellectual Property Counsel, CENTERPULSE USA INC., 3 East Greenway Plaza, Suite 1600, Houston, TX, 77046, US

(21) Appl. No.: 10/262,619

(22) Filed: Oct. 1, 2002

Related U.S. Application Data
(63) Continuation-in-part of application No. 09/605,804, filed on Jun. 28, 2000.
(63) Continuation-in-part of application No. 09/620,056, filed on Jul. 20, 2000.

(51) Int. Cl. A61F 2/00; B01D 3/00

(52) U.S. Cl. 424/423; 203/99

(57) ABSTRACT

Undesired agents, which can reduce biocompatibility, can be selectively and substantially removed from implantable medical devices using methods of the present invention. Pressure and temperature of a supercritical fluid are adjusted to selectively remove one or more undesired agents from an implantable medical device perfused with the supercritical fluid. Treated implantable medical devices comprising at least about 75 wt % less of at least one undesired agent than the same device before undergoing a treatment are also disclosed.

### BACKGROUND OF THE INVENTION

This application is a continuation-in-part application of co-pending U.S. patent application Ser. Nos. 09/605,804, filed Jun. 28, 2000 and 09/620,056, filed Jul. 20, 2000, both of which are hereby incorporated by reference herein in their entirety.

### FIELD OF THE INVENTION

The present invention relates generally to the fields of implantable medical devices. More particularly, it concerns the use of supercritical fluids for treating implantable medical devices to improve the biocompatibility of the devices.

### DESCRIPTION OF RELATED ART

Implantable medical devices have become critical in the management of a variety of human diseases and other conditions. The implantable medical devices can comprise polymers, metals, ceramics, and animal tissues. Examples of such devices can include heart valves, sewing cuffs, vascular grafts, pacemaker leads, medical tubing, fabric patches, catheters, catheter cuffs, annuloplasty rings, coronary stents, peripheral stents, femoral prostheses, acetabular prostheses, dental prosthesis, and orthopedic prostheses, among others.

Antistatic agents and friction reducing agents can be used in processing polymeric components of implantable medical devices, while polishing compounds can be used in processing ceramic components of implantable medical devices. Thus, finished implantable medical devices can comprise such processing aids, as well as dust particles that are introduced during processing. It is believed that the dust particles, residual processing agents (e.g., antistatic agents or friction reducing agents, among others), organic contaminants, and/or certain relatively low-molecular weight compounds, when introduced into an implantable medical device during its manufacture, and present in or on the finished device, can contribute to reduced biocompatibility of the device upon implantation into a patient. These undesired agents are thought to have the potential to cause an inflammatory response in the patient, or to have cytotoxic effects on patient tissue. In order to minimize the chance of complications, it is desirable to produce medical devices comprising lower levels of undesired agents (e.g., processing agent and dust particles, among others) for implantation into patients.

### SUMMARY OF THE INVENTION

In one embodiment, the present invention is directed to a method of treating an implantable medical device. The method comprises perfusing an implantable medical device with a supercritical fluid. The device comprises at least one undesired agent, such as processing aids, dust particles, organic contaminants or low-molecular weight compounds, among others. Perfusing the implantable medical device involves contacting the medical device with the supercritical fluid and removing at least a portion of the undesired agent from the implantable medical device. The supercritical fluid and the removed portion of the undesired agent are then separated from the medical device. The method can further comprise additional steps. In one embodiment the supercritical fluid used in the method is supercritical carbon dioxide, and the perfusing step and the separating step are repeated at least one time.

In another embodiment, the present invention is directed to an implantable medical device that has been treated with a supercritical fluid. In preferred embodiments, the treated medical device comprises at least about 75 wt % less of at least one undesired agent than the same device before undergoing the treatment. The implantable medical device preferably comprises at least one material selected from the group consisting of polymers, metals, and ceramics, and in certain embodiments the device further comprises fixed (i.e., crosslinked) animal tissues.

Using methods and compositions of the present invention can result in medical devices having improved biocompatibility with patient tissue, when the medical device is implanted in a patient.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a cross-sectional view of an annuloplasty ring suitable for treatment according to the present invention.

FIG. 2 is a process flow diagram for one embodiment of the invention.

### DESCRIPTION OF ILLUSTRATIVE EMBODIMENTS

A substance becomes a supercritical fluid above its critical point of temperature and pressure. A supercritical fluid maintained above its critical temperature cannot be liquefied regardless of the pressure applied. Critical pressure is the pressure required to liquefy a supercritical fluid at its critical temperature.

A supercritical fluid is a single phase of a substance exhibiting physicochemical properties intermediate between those of liquids and vapors. Characteristics of a supercritical fluid include: dense gas properties, solubilities approaching liquid phase, and diffusivities approaching gas phase. Dynamic viscosities of supercritical fluids approach those of the normal gaseous state. Above the critical point (e.g., critical temperature and critical pressure), but still close to the critical point, the diffusion coefficient of a supercritical fluid is more than ten times that of a liquid. Changes in viscosity and diffusivity are more pronounced at temperatures and pressures above and close to the critical point. Mass transfer is rapid in supercritical fluids.

The density, viscosity and diffusivity of a supercritical fluid depend on both temperature and pressure. Of course, for a fluid to remain supercritical, it must be maintained above its critical point. Thus, to adjust the solvating-power, density, viscosity, and diffusivity of a supercritical fluid, temperatures and pressures are modified above the critical point of the supercritical fluid. Solvating power of a supercritical fluid is, for example, preferably adjusted by changing the pressure. Increasing the pressure will increase the density of the supercritical fluid. Supercritical fluids that can remove easily extracted materials while the fluid is maintained at a low density, can often also remove materials that are more difficult to extract by raising the pressure- and therefore the density- of the supercritical fluid. Since temperature and pressure are interrelated, changing the temperature of a supercritical fluid above the critical temperature will also change the pressure and density of the supercritical fluid.

Implantable medical devices which can be treated by methods of the present invention can be selected from the group consisting of heart valves, sewing cuffs, vascular grafts, pacemaker leads, medical tubing, fabric patches, catheters, catheter cuffs, annuloplasty rings, coronary stents, peripheral stents, femoral prostheses, acetabular prostheses, dental prostheses, and orthopedic prostheses, among others. Implantable medical devices of the present invention comprise at least one manufactured component. For example a heart valve (e.g., an implantable medical device) can comprise a manufactured component that is a metal stent having animal tissue applied over it to form leaflets. Another example is a vascular graft that comprises a polymeric sleeve and animal vascular tissue inserted in the sleeve, wherein the polymeric sleeve is a manufactured component. Yet another example is a heart valve comprising a metal base and polymeric leaflets, wherein both components are manufactured.

Such implantable medical devices of the present invention can comprise at least one material selected from the group consisting of polymers, metals, and ceramics. Preferably, the material or materials that are part of the structure of the implantable medical device can be safely treated with a supercritical fluid using methods of the present invention, such that the structural integrity of the device is maintained (e.g., preferably the material(s) that comprise the device remains essentially undissolved by the supercritical fluid under the conditions used for treating the medical device and/or preferably essentially none of the material is removed from the device after it has been perfused with the supercritical fluid). Preferably, less than about 0.1 wt % of a structural material is removed or dissolved by the supercritical fluid treatment. Thus, for example, if the implantable medical device is polymeric, the supercritical fluid of the treatment will preferably dissolve essentially none of the polymer or remove essentially none of the polymer (e.g., less than about 0.1-3.0% by weight removed) from the medical device, depending upon the chemical structure and the purity of the polymer used to fabricate the device.

In certain embodiments, the implantable medical device comprises a polymer (e.g., a Dacron sewing cuff, among others). The polymer can comprise any polymer known in the art for making implantable medical devices. Preferably the medical device comprising a polymer comprises at least one polymer selected from the group consisting of rubber, polyester (e.g., polyethylene terephthalate), polyethylene, polyurethane, silicone rubber, polytetrafluoroethylene, and latex, among others. Polyethylene terephthalate and polytetrafluoroethylene are particularly preferred.

In certain embodiments, an implantable medical device that comprises a polymer can optionally further comprise additional components comprising metal, ceramic, or animal tissue. For example, an implantable medical device of the present invention can comprise a heart valve having a polymeric body and a metal stent. Alternatively, an implantable medical device of the present invention can comprise a polymeric sleeve surrounding a tubular fixed vascular or pericardial tissue from an animal, wherein the sleeve and the animal tissue are used as a vascular graft. In another example, an endoprothesis can comprise a fixing stem of fiber reinforced plastic having a ceramic head. Preferably, when such other components are part of the medical device comprising a polymer they can be safely treated with a supercritical fluid using methods of the present invention, such that the structural integrity of the medical device is essentially intact after treatment.

In certain embodiments an implantable medical device of the present invention comprises a metal (i.e., stents, among others). The metal can be any metal known in the art for use in implantable medical devices. The term “metal” as used in the present application is used to refer both to relatively pure metals and metal alloys. A preferred metal for use in implantable medical devices is titanium or titanium alloys. Precious metal alloys (e.g., gold, silver, or iridium, among others) can also be used in certain implantable devices, especially in dental prostheses. Other metals that can be used in implantable devices include steel and cobalt-chromium alloys, among others. The implantable medical devices of the present invention comprising metal, can, in certain embodiments, further comprise polymer, ceramic, and/or animal tissue components. For example, an orthopedic prosthesis (i.e., for implantation in the femur) can comprise a metal shaft with a joint ball made from ceramic or metal. In another example, a metal orthopedic prosthesis can comprise a polymeric or a polished ceramic surface at the articulating interface. In yet another example, tissue from a porcine heart valve can be mounted on a metal stent using known methods and subsequently implanted in a patient. In certain embodiments the animal tissue has been fixed by methods known in the art. Preferably, when such other components are part of the medical device comprising a metal they can be safely treated with a supercritical fluid using methods of the present invention, such that the structural integrity of the medical device is essentially intact after treatment.

The implantable medical devices of the present invention comprising ceramic (e.g., hydroxyapatite, pyrolytic carbon, among others in cancellous and noncancellous configurations), can, in certain embodiments, further comprise polymer, metal, and/or animal tissue components. As described above, an endoprothesis can comprise a fixing stem of fiber reinforced plastic having a ceramic head, and an orthopedic prosthesis can comprise a metal shaft with a joint ball made from ceramic or metal. Preferably, when such other components are part of the medical device comprising a ceramic they can be safely treated with a supercritical fluid using methods of the present invention, such that the structural integrity of the medical device is essentially intact after treatment.

Medical devices treated by methods of the present invention comprise at least one undesired agent. As described above, the undesired agent can comprise dust particles, residual processing agents, organic contaminants and/or certain relatively low-molecular weight compounds introduced into an implantable medical device during its manufacture. The dust particles can be particulates, fibers, or shavings, and the dust particles can, for example, comprise soot, dirt, metal, ceramic, pyrolytic carbon, and plastic, among others. Preferably the dust particles are soluble in the supercritical fluid or they can be suspended in the supercritical fluid of the treatment methods of the present invention. An example of a plastic dust particle is a silicone-based contaminant. Typically dust particles that are to be removed from medical devices by methods of the present invention are found at the surface of the medical device.

Undesired agents can be processing agents, and a medical device will comprise different processing agents depending on the materials from which it is made. The processing agents can be any known in the art that are used to produce medical devices. Preferably the processing agents are soluble in the supercritical fluid treatment methods of the present invention. Processing agents that can be undesired agents can be selected from cutting fluids, mold releasers, antistatic agents, friction reducing agents, and polishing compounds, among others. Specific examples of cutting fluids include: mineral oil, oil/water emulsion, ethanolamine, triethanolamine, borate, carboxylic acid, and amide derivatives among others. Examples of mold releasers include: parafilm, sodium silicate, and polytetrafluoroethylene (Teflon), among others. Thus, for example, a molded polymeric implantable medical device can comprise a mold releaser, such as N,N′-ethylene bis(stearamide). Mold releasers used in processing molded or extracted polymeric medical devices are often waxes.

Plasticizing agents can comprise di octyl phthalate (DOP), di iso butyl phthalate (DIBP), di octyl phthalate- food grade (DOG-FG), butyl benzyl phthalate (BBP), di iso octyl phthalate (DIOP), tri octyl tri mellitate (TOTM), di iso decyl phthalate (DIDP), 2 ethyl hexyl acetate (2EHAC), di octyl adipate (DOA), tri iso decyl tri mellitate (TIDTM), di iso decyl adipate (DIDA), di octyl azelate (DOZ), di octyl sebacate (DOS), di octyl terephthalate(DOTP), di butyl maleate (DBM), di octyl nylonate (DON), di butyl phthalate (DBP), and di ethyl oxalate (DEO), among others. Friction reducing agents can comprise the same materials as mold release agents.

Examples of extrusion aids include stearic acid and palmitic acid, among others, while examples of antistatic agents include alkoxylated alkanolamide, lauryl diethanolamide, alkoxylated alkanolamide, and alcohol phosphate, among others. Examples of polishing compounds include diamond, corundum, garnet, emery, quartz, silicon carbide, aluminum oxide, boron carbide, fused and unfused alumina, among others.

Specific examples of processing agents that can be undesired agents, particularly in manufacturing mechanical heart valves, are isopropanol, ethanol, rust inhibitors applied to tools or molds (e.g., CRC), dielectric fluid, heptane, hexane, mineral spirits, coolant (e.g., Master Chemical Trim C-210, Vita Edge, Syntilo 9951), non-ferrous deburring compounds (e.g., AE-11L Compound, Roto Brite Compound), and cleaning compounds (e.g., Bruelin detergent, Oakite BCR cleaning compound, and Chem Crest soap), among others.

Organic contaminants that can be undesired agents can be introduced by the machines used in processing or through human contact with the devices. Preferably the organic contaminants are soluble in the supercritical fluid treatment methods of the present invention. Organic contaminants can be selected from the group consisting of skin oils, machine oils, and pump oils, among others. For example, fingerprints can result in the introduction of skin oils onto a medical device as it is handled.

Low-molecular weight compounds (e.g., molecular weight less than about 500, more preferably molecular weight less than about 260, and more preferably molecular weigh less than about 120) that can be undesired agents can be selected from unreacted monomers, side-reaction products, and catalyst, among others. As an example, a medical device that comprises polyurethane can comprise unreacted monomer (e.g., 4,4′-methylenediphenyl isocyanate; 1,4-butanediol; or polytetramethylene glycol), polymerization catalyst (e.g., dibutyltin dilaurate), and products of side reaction (e.g., cyclic compounds), all of which can be undesired agents.

When the device further comprises animal tissue in addition to ceramic, metal, and/or polymeric components the undesired agent can further be selected from residual fixative, residual fat and fatty acids, aliphatic carboxylic acids which are generally found in natural fats and oils in esterified form, and dead cell remnants.

At least one undesired agent can be at the surface of the implantable medical device (i.e., polishing compounds used on the surface of ceramics, or metal dust particles on the surface of implantable prosthesis) or the undesired agent can be incorporated into (e.g., located within the structure of) the device (i.e., antistatic agent that has been blended into a polymer, unreacted monomer in a polymer, low molecular weight compounds that have permeated a porous ceramic). In the present application, when undesired agents are at the surface of, permeate through, collect in, adhere to, are incorporated into, or otherwise associated with an implantable medical device, the implantable medical device is said to comprise them.

Supercritical fluids and near super-critical fluids (e.g., the pressure and temperature of the fluid are within about 5% of the critical level) used in the present invention can be any known in the art, and they can be substantially comprised of one or more compound. The implantable medical device is contacted with the supercritical fluid for a duration and under conditions of temperature and pressure (e.g., temperature and pressure above critical point), effective to cause removal of at least a portion of at least one undesired agent. In one embodiment, the supercritical fluid used to perfuse the medical device is maintained at a temperature between about 26.5° C. and about 50° C. and at a pressure of between about 2800 psi and about 6500 psi. Of course, the optimal time for contact between the supercritical fluid and the implantable medical device that is being treated will vary depending on a number of parameters, such as the specific supercritical fluid being used and contact temperature and pressure, all of which can be readily determined by one skilled in the art. Thus, in certain embodiments the perfusing step can be carried out for between about 30 seconds to about 7 days, preferably for about 30 to 60 minutes.

Preferably the supercritical fluids and near-supercritical fluids of the present invention are such that they do not damage the implantable medical device's structural materials (e.g., polymers, metals, ceramics, and animal tissues) at the temperatures and pressures at which they are supercritical and that permit them to aid in removal of undesired agents. Preferably the supercritical fluid used in the treatment is allogenic. Furthermore, the supercritical fluid is preferably readily spread over and/or permeates into the structural materials of an implantable medical device to which it is applied. The degree to which a supercritical fluid is able to do this can in part be affected by surface tension effects of solvent components and by the surface characteristics of the material to which it is being applied.

Preferred supercritical fluids of the present invention comprise supercritical CO2. Although any number of supercritical fluids can be used in treatments of the present invention, supercritical fluids comprising supercritical CO2 (SCO2) can confer several advantages on the treatments. SCO2 is insoluble in water, but can be a powerful solvent for lipids, oils, and other small molecular weight organic compounds. Furthermore SCO2 is not a solvent for certain structural materials from which implantable devices can be made (i.e., polytetrafluoroethylene, silicone rubber, and polyethylene terephthalate, among others). Carbon dioxide itself is relatively environmentally friendly, and therefore solvent disposal costs for treatments involving SCO2 are relatively inexpensive. The viscosity of fluids comprising SCO2 can be relatively low, thereby facilitating rapid perfusion of implantable medical devices. Furthermore, a nonspecific precipitation of undesired agent solutes in a recovery apparatus can be obtained through general reduction of pressure or a sufficiently large solvent temperature reduction. Still further, the portion of any supercritical fluid comprising SCO2 is relatively easy to recover, thus reducing processing costs.

The solvating power of a supercritical fluid can be adjusted using known methods, such as through changes in temperature and/or pressure (particularly pressure) of the supercritical fluid. These changes are preferably performed above the critical point of the supercritical fluid, so that the substance remains supercritical. It follows that heating or cooling can be selectively used to remove and recover undesired agent(s) from implantable medical devices. Supercritical fluid introduced by perfusion within and around an implantable medical device can selectively remove (e.g., transport away, for example, in a dissolved or suspended state) one or more undesired agents. Further, selective recovery of undesired agents from supercritical fluid previously removed from an implantable medical device can then be achieved through reduction of solvent pressure (or temperature), which causes precipitation of solute loads. Iterative applications of such a supercritical fluid can be made to selectively remove undesired agents from an implantable medical device.

When necessary, a supercritical fluid of the present invention can additionally comprise one or more cosolvents (e.g., nitrous oxide or ethanol, among others) and/or surfactants (e.g., polysorbate 80 or dipalmitoyl lecithin, among others). Cosolvents and surfactants can be any known in the art that are used with supercritical fluids. Cosolvents can be used in supercritical fluids to modify the ability of the supercritical fluid to dissolve certain compounds. Preferably the cosolvent enhances the fluid's solvating power (e.g., by modifying the polarity or acidity of the supercritical fluid) and therefore its ability to dissolve and remove at least one undesired agent. Thus, cosolvents can aid in removal of otherwise insoluble undesired agents from the implantable medical device. Preferred cosolvents are selected from C1 to C6 alcohols (e.g., methanol, ethanol, etc.), C1 to C6 ethers (i.e., tetrahydrofuran), C1 to C6 aldehydes, aprotic heterocyclics (e.g., n-methyl pyrrolidinone, dimethyl sulfoxide, dimethyl formamide, etc.), acetonitrile, and acetic acid. Surfactants can be used to adjust the solvating power of the supercritical fluid and/or to modify its surface tension.

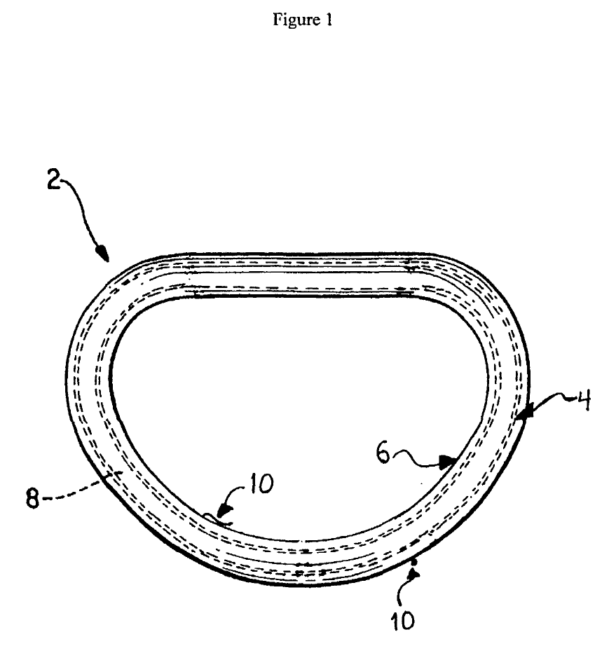

An embodiment of the present invention is directed to a method of treating an implantable medical device. The method comprises perfusing an implantable medical device that comprises at least one undesired agent with a supercritical fluid. Thus, for example, an annuloplasty ring 2 comprising a hollow core 8 as depicted in FIG. 1 can be perfused with a supercritical fluid. The annuloplasty ring 2 comprises an undesired agent which can, for example, comprise dust particles 10 at the surface 6 of the annuloplasty ring or the undesired agent can comprise a processing agent, such as an antistatic agent, which has been incorporated into a polymeric wall 4 of the annuloplasty ring 2.

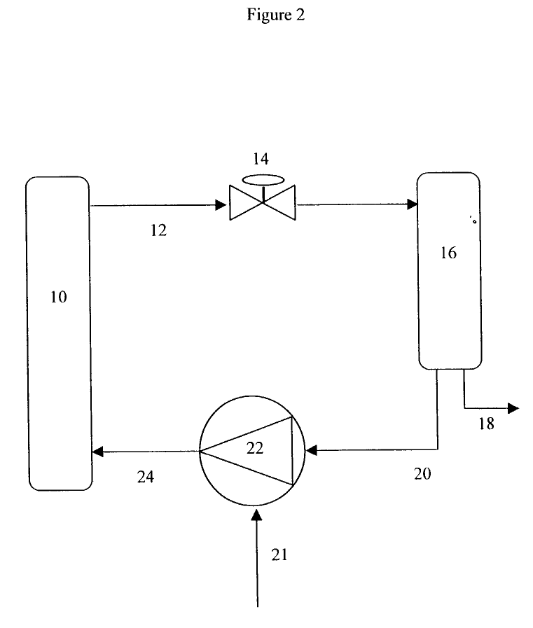

One method of the present invention can be better understood by referencing FIG. 2. An implantable medical device comprising at least one undesired agent can be perfused with a supercritical fluid in vessel 10, such that at least a portion of the undesired agent is removed from the implantable medical device. The removed portion of the undesired agent can in certain embodiments be dissolved in the supercritical fluid, while in other embodiments the undesired agent remains undissolved but is dislodged from the medical device by the supercritical fluid (i.e., removed undesired agent is suspended in the supercritical fluid). The supercritical fluid and the removed undesired agent 12 are separated from the medical device, which remains in vessel 10. A pressure reducing valve and/or a cooler 14 can be used to reduce the pressure and/or temperature of the supercritical fluid such that any undesired agent dissolved in the supercritical fluid is precipitated.

In the separator vessel 16, the supercritical fluid 20 can be separated from undesired agent which had previously been dissolved or suspended in the fluid and which remains in the vessel 16 after separation or that is removed as a waste stream 18 after separation. Thus, the method can comprise recovering the supercritical fluid. Recovering the supercritical fluid can comprise (a) adjusting (e.g., increasing or decreasing) at least one of the temperature or the pressure of the separated supercritical fluid and the removed portion of the undesired agent such that undesired agent is precipitated from the supercritical fluid, and (b) separating the supercritical fluid from the precipitated undesired agent thereby recovering the supercritical fluid.

In certain embodiments, especially those in which the undesired agent is capable of being dissolved in the supercritical fluid (e.g., when the temperature and/or the pressure of the supercritical fluid is adjusted so that the supercritical fluid has the necessary solvating power), the method further comprises adjusting at least one of a pressure or a temperature of the supercritical fluid before perfusing the medical device. The supercritical fluid that is used to perfuse the medical device can be fresh supercritical fluid from makeup stream 21 or it can be supercritical fluid 20 that has been used in previous rounds of treatment.

If the supercritical fluid has been recycled, 20, additional components, such as cosolvents or surfactants, can be added through makeup stream 21 to permit removal of different undesired agents than those which have been removed in a previous round of treatment. Thus, the recycled supercritical fluid 20 or newly introduced supercritical fluid or supercritical fluid components in makeup stream 21 can have their temperature and/or pressure adjusted by a recycle compressor 22 so that the resulting supercritical fluid 24, preferably having increased pressure, can be used to perfuse the medical device in vessel 10. Thus, the temperature/pressure adjusted supercritical fluid 24 can dissolve at least a portion of the undesired agent during the perfusing step in vessel 10 and a portion of the undesired agent can be removed from the medical device.

The process including the adjusting step, the perfusing step, the separating step, the recovering step and the returning step, described above, can be repeated as necessary until substantially all undesired agent is removed from the medical device. In a preferred embodiment, the supercritical fluid used in the process is supercritical carbon dioxide, and the perfusing step and the separating step are repeated at least one time in order to remove more of an undesired agent than was accomplished by the first round. Preferably the perfusing and the separating steps performed with the supercritical carbon dioxide are repeated until substantially all (e.g., about 99 wt %) of the undesired agent is removed.

In one example of an embodiment of the present invention, the pressure and/or temperature of the supercritical fluid can be adjusted so that as a medical device is perfused, the supercritical fluid dissolves at least a portion of a first undesired agent (i.e., an antistatic agent). The supercritical fluid and the removed portion of the first undesired agent are separated from the medical device. The pressure of the supercritical fluid is adjusted again so that the undesired agent that had been dissolved in the fluid is precipitated. The supercritical fluid has its temperature and/or pressure adjusted so that it can dissolve at least a portion of a second undesired agent that the medical device comprises (i.e., a polishing compound) having a different solubility than the first undesired agent. The supercritical fluid that has had its temperature and/or pressure readjusted for another round of removal is used to perfuse the medical device again, and this time at least a portion of the second undesired agent is dissolved by the fluid and removed. The second undesired agent can be precipitated from the supercritical fluid, and the supercritical fluid can be used to treat the same medical device or another medical device to remove additional undesired agent.

In another example of a process of the present invention, the temperature and/or pressure of the supercritical fluid is adjusted before it is used to perfuse a medical device, and the supercritical fluid is capable of dissolving at least a portion of at least two different undesired agents. The supercritical fluid and the removed portion of undesired agent is separated from the medical device, and the undesired agents dissolved in the supercritical fluid are selectively precipitated from the supercritical fluid by appropriate control of the fluid's temperature and pressure.

Selective precipitation of undesired agent(s) from a supercritical fluid can be effected, for example, by either heating or cooling such fluids (depending on the solutes), and/or by decreasing solvent pressure sufficiently to cause reversion of a solvent component to a subcritical state. In preferred embodiments, supercritical fluids comprising SCO2 can further comprise a supercritical cosolvent, such as nitrous oxide. In such embodiments, carbon dioxide and nitrous oxide solvent components can be converted from supercritical to subcritical states simultaneously or sequentially to effect selective recovery of undesired agent(s). Preheated or precooled portions of recovery apparatus can thus be made to preferentially recover one or more selected undesired agent(s).

For example, reduction of solvent temperature below 36.5° C. will render any nitrous oxide present subcritical, thus generally reducing its solvating power. Similarly, reduction of solvent temperature below 31.3° C. will render any carbon dioxide present subcritical, with an analogous reduction in its solvating power. Thus in certain embodiments in which the supercritical fluid comprises carbon dioxide, the supercritical fluid is preferably maintained above a temperature of about 31.3° C. during the perfusing step, and more preferably above a temperature of about 45° C. Furthermore in certain embodiments in which the supercritical fluid comprises carbon dioxide and nitrous oxide, the supercritical fluid is preferably maintained above a temperature of about 36.5° C. during the perfusing step.

Preferably an implantable medical device that has undergone methods of treatment of the present invention comprises at least about 75 wt % less of at least one undesired agent than the same device before undergoing treatment. More preferably the treated implantable medical device comprises at least about 90 wt % less of at least one undesired agent than the same device before undergoing treatment, and most preferably the treated implantable medical device comprises at least about 99 wt % less of at least one undesired agent than the same device before undergoing treatment. The following examples are included to demonstrate preferred embodiments of the invention. The examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1.

Pyrolite(r) carbon mechanical heart valve leaflets, after going through a detail polishing process, were handled by bare hands to determine if supercritical fluid cleaning could remove the deposited finger oil. The leaflets were dehydrated prior to supercritical fluid cleaning: 1) soaked in 10 ml of 50% (v/v) ethanol (USP Ethyl Alcohol, Pharmco Products Inc., Brookfield, Conn.) in deionized water for 1 minute, 2) soaked in 10 ml of 70% (v/v) ethanol in deionized water for 1 minute, 3) soaked in 10 ml of 85% (v/v) ethanol in deionized water for 1 minute, 4) soaked in 10 ml of 95% (v/v) ethanol in deionized water for 5 minutes, and finally, 5) soaked in 10 ml of 100% ethanol for 15 minutes. It should be noted that after the ethanol was first opened, molecular sieve (Grade 564 3A effective pore size 8-12 mesh beads, code # 56408080237, Davison Chemical, Baltimore, Md.) was added to the bottle to keep the solvent dry; therefore, the ethanol was passed through a 0.2 μm filter (Gelman Acrodisc(r) CR PTFE, cat # 4225, Pall Life Sciences, Ann Arbor, Mich.) prior to use.

After dehydration, the leaflets were placed between 21 mm Wavy Washers (cat # 8767-01, Tousimis Research Corporation, Rockville, Md.) and subsequently into the Cover Slip Holder (cat # 8767, Tousimis Research Corporation, Rockville, Md.). The whole apparatus was soaked in 100% ethanol for 15 minutes, and then allowed to dry for 15 minutes on Kimwipe(r)EX-L wipes (cat # 34155, KimberlyClark Corporation, Roswell, Ga.).

The leaflets were then cleaned with supercritical carbon dioxide (CO2) following the procedure written in the Installation and Operation Manual for a Tousimis Samdri-780A Critical Point Dryer. All of the valves (Inlet, Cool, Bleed, and Purge/Vent) were closed. The high-pressure hose, water/oil filter (cat # 8782, Tousimis Research Corporation, Rockville, Md.), and the particulate filter (cat # 8781, Tousimis Research Corporation, Rockville, Md.) were connected between the Samdri-780A and the compressed CO2 cylinder (mounted on a floor scale to monitor supply).

With CO2 off, the power and lamp were turned on. The Cover Slip Holder with the leaflets was placed inside the chamber with enough 100% ethanol to cover them and then the chamber was sealed. The valve on the CO2 cylinder was opened. The Cool valve was opened just long enough to cool the chamber down to 0° C. Next, the Inlet valve was slowly opened until the chamber completely filled with liquid CO2. Then the Inlet valve was opened fully. While keeping the temperature below 12(C, the ethanol was purged from the chamber by slowly opening the Purge-Vent valve, which was closed afterwards.

Once the chamber completely filled with CO2, the Inlet and the cylinder valves were closed. The Heat switch was turned on to allow the chamber temperature to rise. The temperature and pressure were recorded approximately every minute over a 15-minute time period. The temperature ranged from 4(C to 37(C, while the pressure ranged from 850 psi to 1325 psi. The system was allowed to remain at “equilibrium state” above the critical temperature (31(C) and pressure (1100 psi) for more than 4 minutes. The Bleed valve was opened so that the pressure decreased approximately 100 psi per minute. Once the pressure reached 250 psi, the Bleed valve was opened fully. The Cover Slip Holder with leaflets was removed from the chamber for evaluation and all the valves, heater, lamp, and power were turned off. No fingerprints or other residues were observed upon visual inspection of the leaflets.

### EXAMPLE 2.

Pyrolite leaflets, after going through a general polishing process, were processed by the same procedure stated above in Example 1 to determine if supercritical fluid cleaning could remove the remaining residues. Over a 15 minute time period, the temperature ranged from 4(C to 43(C, while the pressure ranged from 800 psi to 1350 psi. The system was maintained at “equilibrium state” above the critical temperature (31(C) and pressure (1100 psi) for more than 5 minutes before shutting down the system. No fingerprints or other residues were observed upon visual inspection of the leaflets.

### EXAMPLE 3.

Silicone elastomer leaflets, cut from a cuffed elastomer valve, were processed by the procedure stated above to determine if supercritical fluid cleaning could remove the lint left by the sewing cuff attachment procedure. Over a 15 minute time period, the temperature ranged from 4(C to 44(C, while the pressure ranged from 800 psi to 1350 psi. The system was maintained at “equilibrium state” above the critical temperature (31(C) and pressure (1100 psi) for more than 5 minutes before shutting down the system. No finger prints were observed upon visual inspection of the leaflets, but some contaminant lint fibers were observed. However at least a 50% reduction of contaminant fibers was observed after a single treatment with supercritical carbon dioxide. It is anticipated that such remaining contamination on the leaflets can be further reduced or eliminated completely by doing additional rounds of treatment with supercritical fluid.

All of the methods and devices disclosed and claimed herein can be made and executed without undue experimentation in light of the present disclosure. While the devices and methods of this invention have been described in terms of preferred embodiments, it will be apparent to those of skill in the art that variations may be applied to the devices and methods and in the steps or in the sequence of steps of the methods described herein without departing from the concept, spirit and scope of the invention. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating an implantable medical device, comprising:
providing an implantable medical device comprising at least one undesired agent;
perfusing the implantable medical device and undesired agent with a supercritical fluid such that at least a portion of the undesired agent is removed from the implantable medical device; and
separating the supercritical fluid and the removed portion of the undesired agent from the medical device.

2. The method of claim 1, wherein the undesired agent is soluble in the supercritical fluid, and wherein the method further comprises adjusting at least one of a pressure or a temperature of the supercritical fluid before perfusing the medical device, such that the supercritical fluid dissolves at least a portion of the undesired agent in the perfusing step, thereby removing a portion of the undesired agent from the medical device.

3. The method of claim 2, wherein the method further comprises recovering the supercritical fluid by (a) adjusting at least one of the temperature or the pressure of the separated supercritical fluid and the removed portion of the undesired agent such that the undesired agent is precipitated from the supercritical fluid, and (b) separating the supercritical fluid from the precipitated undesired agent, thereby recovering the supercritical fluid.

4. The method of claim 3, wherein the method further comprises returning the recovered supercritical fluid to the adjusting step before perfusing the medical device, and wherein the adjusting step, the perfusing step, the separating step, the recovering step and the returning step are repeated as necessary until substantially all undesired agent is removed from the medical device.

5. The method of claim 1, wherein the supercritical fluid is supercritical carbon dioxide, and wherein the perfusing step and the separating step are repeated at least one time.

6. The method of claim 1, wherein the medical device is selected from the group consisting of heart valves, sewing cuffs, vascular grafts, pacemaker leads, medical tubing, fabric patches, catheters, catheter cuffs, annuloplasty rings, coronary stents, peripheral stents, femoral prostheses, acetabular prostheses, dental prosthesis, and orthopedic prostheses.

7. The method of claim 1, wherein the medical device comprises at least one material selected from the group consisting of polymers, metals, and ceramics.

8. The method of claim 7, wherein the medical device comprises at least one polymer selected from the group consisting of rubber, polyester, polyethylene, polyurethane, silicone, polytetrafluoroethylene, and latex.

9. The method of claim 1, wherein the supercritical fluid comprises carbon dioxide.

10. The method of claim 9, wherein the supercritical fluid is maintained at a temperature above about 31.3° C. during the perfusing step.

11. The method of claim 9, wherein the supercritical fluid is maintained at a temperature above about 45° C. during the perfusing step.

12. The method of claim 1, wherein the supercritical fluid further comprises at least one of a cosolvent or a surfactant.

13. The method of claim 12, wherein the cosolvent is selected from the group consisting of C1 to C6 alcohols, C1 to C6 ethers, C1 to C6 aldehydes, aprotic heterocyclics, acetonitrile, and acetic acid.

14. The method of claim 12, wherein the supercritical fluid comprises carbon dioxide and wherein the cosolvent comprises nitrous oxide, and wherein the supercritical fluid is maintained at a temperature above about 36.5° C. during the perfusing step.

15. The method of claim 1, wherein the undesired agent is selected from dust particles, organic contaminants, low-molecular weight compounds, and processing aids.

16. The method of claim 1, wherein the perfusing step is carried out for between about thirty seconds and 7 days.

17. The method of claim 1, wherein the perfusing step is carried out for between about 30 minutes and 60 minutes.

18. The method of claim 1, wherein the implantable medical device comprises at least about 75 wt % less of at least one undesired agent after the separating step than the same device before undergoing treatment.

19. The method of claim 1, wherein the implantable medical device comprises at least about 90 wt % less of at least one undesired agent after the separating step than the same device before undergoing treatment.

20. The method of claim 1, wherein the implantable medical device comprises at least about 99 wt % less of at least one undesired agent after the separating step than the same device before undergoing treatment.

21. A treated implantable medical device prepared by a process comprising the steps of:
perfusing an implantable medical device that comprises at least one undesired agent with a supercritical fluid, such that at least a portion of the undesired agent is removed from the implantable medical device; and
separating the supercritical fluid and the removed portion of the undesired agent from the medical device, thereby producing a treated implantable medical device.

22. The implantable medical device of claim 21, wherein the undesired agent is capable of being dissolved in the supercritical fluid, and wherein the method further comprises the step of:
adjusting at least one of a pressure or a temperature of the supercritical fluid before perfusing the medical device, such that the supercritical fluid dissolves at least a portion of the undesired agent in the perfusing step, thereby removing a portion of the undesired agent from the medical device.

23. The implantable medical device of claim 22, wherein the method further comprises the step of:
recovering the supercritical fluid by
(a) adjusting at least one of the temperature or the pressure of the separated supercritical fluid and the removed portion of the undesired agent such that the undesired agent is precipitated from the supercritical fluid; and
(b) separating the supercritical fluid from the precipitated undesired agent to obtain a recovered supercritical fluid.

24. The implantable medical device of claim 23, wherein the method further comprises the steps of:
adjusting at least one of a pressure or a temperature of the recovered supercritical fluid;
perfusing the medical device with the recovered supercritical fluid; and
again recovering the supercritical fluid to obtain a recovered supercritical fluid.

25. The implantable medical device of claim 24, wherein said steps of adjusting at least one of a pressure or a temperature of the recovered supercritical fluid, perfusing the medical device with the recovered supercritical fluid, and again recovering the supercritical fluid to obtain a recovered supercritical fluid are repeated until substantially all undesired agent is removed from the medical device.

26. The implantable medical device of claim 21, wherein the supercritical fluid is supercritical carbon dioxide, and wherein the perfusing step and the separating step are repeated at least one time.

27. The implantable medical device of claim 21, wherein the treated implantable medical device comprises at least about 75 wt % less of at least one undesired agent than the same device before undergoing a treatment

28. The implantable medical device of claim 21, wherein the device comprises at least about 90 wt % less of the undesired agent than the same device before undergoing the treatment.

29. The implantable medical device of claim 21, wherein the device comprises at least about 99 wt % less of the undesired agent than the same device before undergoing the treatment.

30. The implantable medical device of claim 21, wherein the undesired agent is selected from dust particles, organic contaminants, low-molecular weight compounds and processing aids.

31. The implantable medical device of claim 21, wherein the device is selected from the group consisting of heart valves, sewing cuffs, vascular grafts, pacemaker leads, medical tubing, fabric patches, catheters, catheter cuffs, annuloplasty rings, coronary stents, peripheral stents, femoral prostheses, acetabular prostheses, dental prostheses, and orthopedic prostheses.

32. The implantable medical device of claim 21, wherein the medical device comprises at least one material selected from the group consisting of polymers, metals, and ceramics.

33. The implantable medical device of claim 21, wherein the supercritical fluid comprises carbon dioxide.

34. The implantable medical device of claim 218, wherein the supercritical fluid further comprises at least one of a cosolvent or a surfactant.
